(19) United States
(12) Patent Application Publication
(10) Pub. No.: US 2021/0267466 A1
(43) Pub. Date: Sep. 2, 2021

(54) INPLANTABLE DEVICE FOR MEASURING CARDIOLOGY PARAMETERS

(71) Applicant: Kazuo TANI, Osaka-shi, Osaka (JP)

(72) Inventor: Kazuo TANI, Osaka-shi, Osaka (JP)

(21) Appl. No.: 16/972,494

(22) PCT Filed: Jun. 5, 2019
(86) PCT No.: PCT/JP2019/022304

§ 371 Date: Dec. 4, 2020

(30) Foreign Application Priority Data
Jun. 5, 2018 (JP) 2018-107853
Mar. 7, 2019 (JP) 2019-041155

(51) Int. Cl. A61B 5/026; A61B 5/00; A61B 5/0245

(52) U.S. Cl. CPC A61B 5/026; A61B 5/0006; A61B 5/0022; A61B 2560/0214; A61B 5/6876; A61B 5/0245; A61B 5/742

(57) ABSTRACT

A blood flow rate measurement system has a blood flow rate probe and a mobile terminal. The blood flow rate probe is used to measure a blood flow rate of a blood vessel of a test body and convert it to an electric signal. The mobile terminal displays the blood flow rate measured by the blood flow rate probe. The blood flow rate data is transmitted from the blood flow rate probe to the mobile terminal via wireless communication.

### TECHNICAL FIELD

The present disclosure relates to a blood flow rate measurement system for measuring blood flow rates of a blood vessel of a subject such as a human body.

### BACKGROUND

For example, measuring blood flow rates in blood vessels of patients (subjects) is necessary before, during, and after an operation, such as a cardiac operation. Thus, blood flow rates can be measured by a blood flow rate probe. The measurement results may then be displayed to doctors at a time of need, such as during an operation. Japanese Patent No. 2915343 discloses a measurement device for such measurement and display.

### SUMMARY OF THE INVENTION

However, the blood flow rate probe is connected to a display device by a cable for transmitting signals. The blood flow rate probe is held along the blood vessels of the patients. The display device is located in an operation room. Thus, the cables extending between the blood flow rate probe and the display device can become disorganized on the floor of the operation room. This can prevent efficient operation procedures.

Therefore, the blood flow rate measurement system needs to be improved.

In the first aspect of the present disclosure, a blood flow rate measurement system has a blood flow rate probe and a display device. The blood flow rate probe may be used to measure a blood flow rate of a blood vessel of the test body and convert it to an electric signal. The display device may be used to display the blood flow rate measured by the blood flow rate probe. The blood flow rate data is transmitted from the blood flow rate probe to the display device via wireless communication.

According to the first aspect of the present disclosure, the blood flow rate data is transmitted from the blood flow rate probe to the display device via wireless communication. Thus, a signal transmission cord is unnecessary to transmit the signal from the blood flow rate probe to the display device. Thus, the signal transmission cable does not become disorganized on the floor of, for example, an operation room or a laboratory.

The second aspect of the present disclosure is that the blood flow rate probe has a monitor to display a part of the blood flow data in the above-mentioned first aspect.

According to the second aspect of the present disclosure, the part of the blood flow data is displayed on the monitor of the blood flow rate probe. Thus, doctors can check, for example, summaries of the blood flow rate, electrocardiogram data, or their changes easily via the monitor of the blood flow rate probe, at their hands without needing to look at the display device. As a result, the doctors have no need to watch the display device to check the blood flow rate frequently, and can further concentrate on the operation.

The third aspect of the present disclosure is that the blood flow rate probe is embedded and fixed in the test body for a certain period of time in the above-mentioned first aspect.

According to the third aspect of the present disclosure, for example, the blood flow rate and the electrocardiogram can be measured continuously for a certain period of time after the operation by the embedded blood flow rate probe in the test body.

In the fourth aspect of the present disclosure, an internally implantable blood flow rate measurement system has a blood flow rate probe, a display device, a transmitter, and a power source device. The blood flow rate probe is embedded in the test body, attached to a blood vessel of the test body, and enabled to convert a blood flow rate to an electric signal. The display device is located outside the test body and used to display the blood flow rate measured by the blood flow rate probe. The transmitter is used to transmit the blood flow rate data to the display device via wireless communication after receiving the electric signal from the blood flow rate probe. The power source device is used to supply working power to the blood flow rate probe and the transmitter. The power source device is connected to the blood flow rate probe via an electric connection means allowing charging from a distance. Additionally, the power source device is allowed to be located on the surface side of the test body and to be separated from the blood flow rate probe.

According to the fourth aspect of the present disclosure, the power source device may be located on the surface side of the test body, separated from the blood flow rate probe. Thus, if the blood vessel to be measured is located at a narrow space in the body, only the blood flow rate probe can be inserted into the narrow space. Further, the power source device can be placed at a proper location separated from the blood flow rate probe. Furthermore, the power source device may be located on the surface side of the test body. Thus, maintenance of the power source device can be improved.

The fifth aspect of the present disclosure is that the power source device is allowed to be embedded in the test body in a state that the power source device is accommodated in a casing in the fourth aspect. Additionally, the electric connection means is composed of a freely bendable cord.

According to the fifth aspect, the power source device is connected to the blood flow rate probe via the freely bendable cord. Thus, if the blood vessel is located at the narrow space in the body, only the blood flow rate probe can be inserted into the narrow space. Additionally, the power source device can be placed at the proper location separated from the blood flow rate probe. Even if connection distance between the blood flow rate probe and the power source device changes, the cord is freely bendable, and, thus, the cord can flexibly adapt to the change of the distance. Further, the power source device is accommodated in the casing. Thus, the power source device is protected from being exposed in the body.

The sixth aspect of the present disclosure is that the power source device is located outside the test body and has a power supply equipment in the fourth aspect. The power supply equipment is wirelessly fed power by the power source device and supplies the working power to the blood flow rate probe and the transmitter.

According to the sixth aspect, the blood flow rate data measured by the blood flow rate probe is wirelessly transmitted to the display device via the transmitter, and then displayed on the display device. Furthermore, the blood flow rate probe is wirelessly fed power from the power source device located outside of the test body. That is, only the blood flow rate probe is embedded in the test body. This can decrease the physical burden on the test body during the measurement of the blood flow rate.

In the seventh aspect of the present disclosure, the blood flow rate probe used in the internally implantable blood flow rate measurement system of any of the above-mentioned fourth, fifth, or sixth aspects may have a torso member. The torso member is attachable to the blood vessel by wrapping around an outer peripheral surface of the blood vessel to be measured. The torso member has a partially opened circular pipe shape, such that the blood vessel is allowed to be inserted into the circular pipe shape. Additionally, the torso member has a function to measure the blood flow rate of the blood vessel.

According to the seventh aspect, the circular pipe shaped torso member of the blood flow rate probe can be wrapped around the outer peripheral surface of the blood vessel for which the blood flow rate is to be measured. The torso member can also be easily attached to a blood vessel located in a narrow space. In this way, the torso member can be used for various blood vessels.

### BRIEF DESCRIPTION OF THE DRAWINGS

FIG. 1 is a schematic view of a system configuration of the first embodiment of the present disclosure.

FIG. 2 is a system configuration diagram illustrating the first specific example of the first embodiment.

FIG. 3 is a system configuration diagram illustrating the second specific example of the first embodiment.

FIG. 4 is a schematic view of a system configuration of the second embodiment of the present disclosure.

FIG. 5 is a system configuration diagram illustrating the first specific example of the second embodiment.

FIG. 6 is a system configuration diagram illustrating the second specific example of the second embodiment.

FIG. 7 is a schematic view of a system configuration of the third embodiment of the present disclosure.

FIG. 8 is a system configuration diagram illustrating the first specific example of the third embodiment.

FIG. 9 is an enlarged perspective view illustrating a major portion of a blood flow rate probe of the second specific example of the third embodiment.

FIG. 10 is an enlarged perspective view similar to FIG. 9 and illustrates the blood flow rate probe attached to a blood vessel.

FIG. 11 is a schematic view of a system configuration of the fourth embodiment of the present disclosure.

FIG. 12 is a schematic view of a system configuration of the fifth embodiment of the present disclosure.

FIG. 13 is a side view of the blood flow rate probe of the fifth embodiment.

FIG. 14 is a perspective view of the blood flow rate probe shown in FIG. 13.

FIG. 15 is an operation explanatory view of the blood flow rate probe shown in FIG. 13.

FIG. 16 is a side view of the blood flow rate probe of another example of the fifth embodiment.

FIG. 17 is a perspective view of the blood flow rate probe shown in FIG. 16.

FIG. 18 is a schematic view of a system configuration of the sixth embodiment of the present disclosure.

### MODES FOR CARRYING OUT THE INVENTION

### Configuration of the First Embodiment

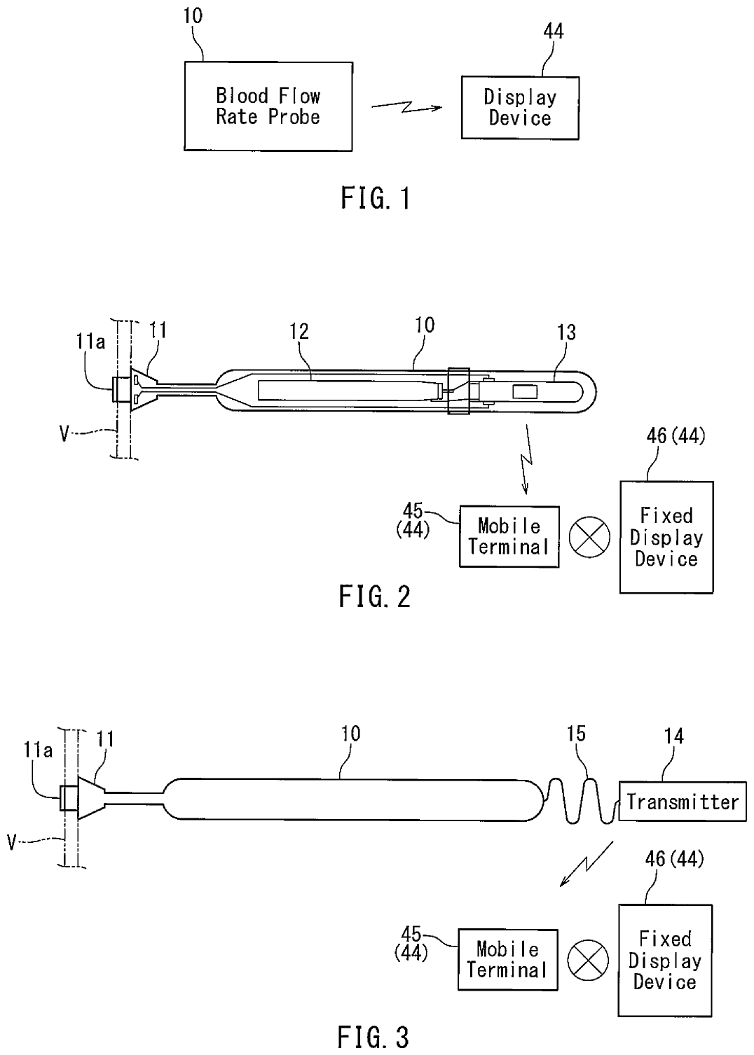

FIG. 1 illustrates the first embodiment of the present disclosure. In the first embodiment, a blood flow rate probe 10 and a display device 44 are included. The blood flow rate probe 10 is used to measure blood flow rates of a blood vessel of a patient (test body) during an operation. The blood flow rate probe 10 then converts the measured blood flow rate to electric signals. The display device 44 may display the measured blood flow rate obtained from the blood flow rate probe 10 to doctors during an operation. The blood flow rate data transmission from the blood flow rate probe 10 to the display device 44 can be performed via wireless communication. Thus, the blood flow rate probe 10 may have a transmitter, and the display device 44 may have a receiver. The transmitter and the receiver are not shown in FIG. 1. In this case, the wireless communication is performed in the operating room. Accordingly, short range communication, such as Bluetooth®, may be adopted as the means of wireless communication.

### Configuration of the First Specific Example of the First Embodiment

FIG. 2 illustrates the first specific example of the first embodiment. The blood flow rate probe 10 may adopts the TTFM (Transit Time Flow Measurement) disclosed in Japanese Patent No. 2915343. The blood flow rate probe 10 has a head portion 11 and a reflection plate 11a at an edge portion of the probe 10. The blood flow rate can be measured by placing the blood vessel V of a patient between the head portion 11 and the reflection plate 11a. In this embodiment, the blood flow rate probe 10 is shown as a perspective diagram. The blood flow rate probe 10 has a transmitter 13 for short range communication built in an edge portion, at an end opposite of the head portion 11 of the blood flow rate probe 10. The blood flow rate probe 10 has a battery 12, which supplies electricity to the blood flow rate probe 10, built in a position on the head portion 11 side from the transmitter 13. Thus, obtained blood flow rate data of the blood vessel of the patient is transmitted from the transmitter 13 of the blood flow rate probe 10.

As illustrated in FIG. 2, a mobile terminal 45, such as mobile phone or a tablet terminal, may be used as the display device 44 in the first specific example. The mobile terminal 45 has a built-in transceiver (not shown), allowing for short range communication as a standard function. Thus, the mobile terminal 45 can receive the blood flow rate data from the transmitter 13 and display the blood flow rate data on the screen of the mobile terminal 45.

A fixed display device 46 may be used as a display device 44, in addition to the mobile terminal 45. The fixed display device 46 can share the blood flow rate information with the mobile terminal 45, for instance via wireless LAN. The fixed display device 46 may display, for example, an electrocardiogram, a body temperature, a blood pressure, and/or a heart rate, which are measured separately, together with the blood flow rate. Both of the mobile terminal 45 and the fixed display device 46 may not necessarily be provided together as the display device 44. Only either the mobile terminal 45 or the fixed display device 46 may be provided. A plurality of the mobile terminals 45 and/or fixed display device 46 may be installed at different places and may intercommunicate with each other via wireless LAN. Further, the transmitter 13 may have a function to communicate with other devices via wireless LAN. In this case, the transmitter 13 may directly communicate with the fixed display device 46 via wireless LAN, without the need for the mobile terminal 45.

### Configuration of the Second Specific Example in the First Embodiment

FIG. 3 illustrates the second specific example of the first embodiment. Compared to the first specific example, while the second specific example is also characterized in that the blood flow rate probe 10 adopts the TTFM disclosed in Japanese Patent No. 2915343, the blood flow rate probe 10 is a commonly used probe having no transmitter 13. Instead, the output of the blood flow rate probe 10 may be connected to a transmitter 14 via a cord 15. The other components of the second specific example are substantially the same as those of the first specific example in the first embodiment. Accordingly, the same symbols are attached to the same elements. Thus, the repetitive explanation for the same elements are omitted.

The transmitter 14 may be fixed to an operating table. The cord 15 has a length just enough to connect the transmitter 14 fixed to the operating table with the blood flow rate probe 10. Thus, the cord 15 does not become disorganized on the floor of the operating room. The transmitter 14 may be used for short range communication similar to the transmitter 13. Thus, short range communication with the mobile terminal 45 is also possible for the transmitter 14 in addition to the first specific example.

### Function and Effect of the First Embodiment

Signal transmission from the blood flow rate probe 10 to the display device 44 is performed via wireless communication. Thus, a signal transmission cable is unnecessary to transmit signals from the blood flow rate probe 10 to the display device 44. Thus, the signal transmission cable does not become disorganized on the floor of the operating room. When the display device 44 is the mobile terminal 45, doctors or ancillary staffs can easily see the mobile terminal 45 during an operation, so that the doctors and/or ancillary staffs can monitor the blood flow rate easily. The fixed display device 46 may also be used to display the blood flow rate as necessary.

### Configuration of the Second Embodiment

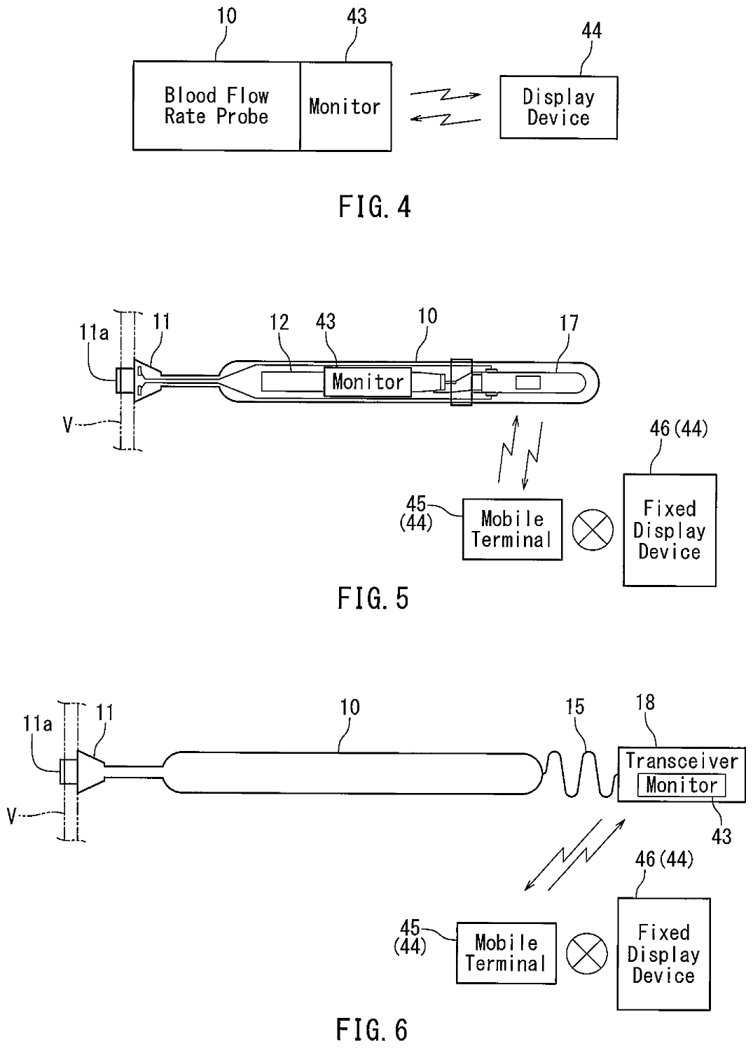

FIG. 4 illustrates the second embodiment of the present disclosure. Compared to the first embodiment, the second embodiment is characterized in that the blood flow rate probe 10 has a monitor 43 to display a part of the data showing the blood flow rate. For display on the monitor 43, the blood flow rate probe 10 has a transceiver to transmit and receive data. Further, the display device 44 also has a transceiver to transmit and receive data. The other respects of the second embodiment are substantially the same as those of the first embodiment. Additionally, the same symbols are attached to the same elements. Thus, repetitive explanations for the same elements are omitted.

The monitor 43 receives a part of the contents displayed on the display device 44. The monitor 43 receives the contents from the display device 44 via the transceiver of the display device 44 and the transceiver of the blood flow rate probe 10. After receiving the contents, the monitor 43 displays it.

### Configuration of the First Specific Example in the Second Embodiment

FIG. 5 illustrates the first specific example of the second embodiment. The blood flow rate probe 10 of this example is basically the same as the blood flow rate probe 10 of the first example of the first embodiment. However, a liquid crystal monitor 43 is attached at a position of the blood flow rate probe 10 where the doctors can easily see the monitor 43 of the blood flow rate probe 10 when operating. Additionally, the transmitter 13 is replaced with a transceiver 17 capable of transmitting and receiving. Configurations and functions of the mobile terminal 45 and the fixed display device 46 are the same as those described in the first example of the first embodiment.

The monitor 43 receives, from a transceiver built into the mobile terminal 45, a part of contents displayed on the mobile terminal 45. The monitor 43 receives the contents via the transceiver 17 of the blood flow rate probe 10. Then, the monitor 43 displays the part of this contents.

### Configuration of the Second Specific Example in the Second Embodiment

FIG. 6 illustrates the second specific example of the second embodiment. Compared to the second specific example in the first embodiment, the transmitter 14 is replaced with a transceiver 18 in this example. However, in other respects, both may be substantially the same.

In this example, the transceiver 18 has the monitor 43, similar to the first specific example of the second embodiment (e.g., see FIG. 5). The monitor 43 receives a part of the contents displayed on the mobile terminal 45 from the mobile terminal 45, via the transceiver 18, and then displays the same.

### Function and Effect of the Second Embodiment

The blood flow rate probe 10 has the monitor 43 configured to display a part of the data corresponding to the blood flow rate. Thus, doctors can check, for example, summaries of the blood flow rate, electrocardiogram data, or any changes thereof on the monitor 43 at hand during an operation. That is, the doctors can check those data easily via the monitor 43 of the blood flow rate probe 10, without needing to look at the mobile terminal 45 or the fixed display device 46. As a result, the doctors have no need to frequently look up the mobile terminal 45 or the fixed display device 46 to check the blood flow rate, and thus can concentrate on the operation.

### Configuration of the Third Embodiment

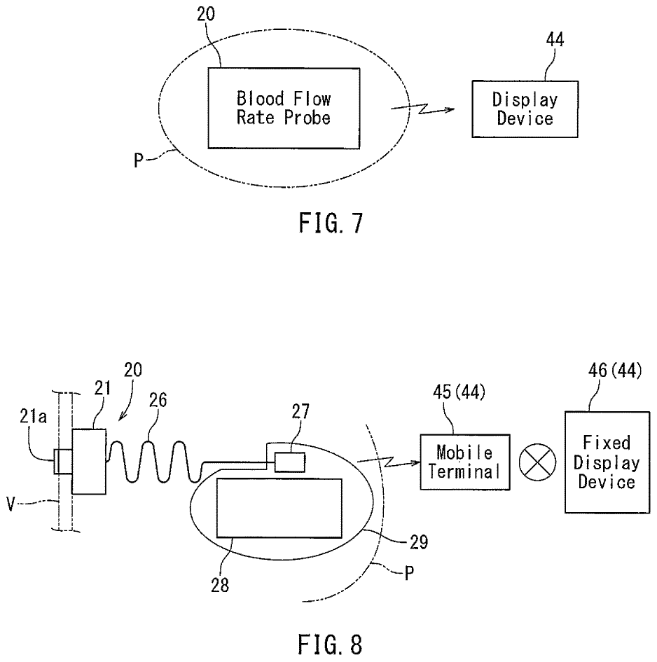

FIG. 7 illustrates the third embodiment of the present disclosure. Compared to the first embodiment, the third embodiment is characterized in that the blood flow rate probe 10 is replaced with a blood flow rate probe 20. The blood flow rate probe 20 is configured to be embedded in the test body (e.g., the patient P). The other aspects of the third embodiment are substantially the same as that of the first embodiment. Thus, the repetitive explanations for the same elements are omitted.

### Configuration of the First Specific Example in the Third Embodiment

FIG. 8 illustrates the first specific example of the third embodiment. The blood flow rate probe 20 has a blood flow rate probe body 21, a transmitter 27, and a cord 26. The blood flow rate probe body 21 and the transmitter 27 may be connected electrically by the cord 26. The blood flow rate probe 20 has basic functions which are the same as those of the blood flow rate probe 10 of the first embodiment (e.g., see FIG. 2). The blood flow rate probe body 21 adopts the TTFM. The blood vessel V located in the patient P is placed between the probe body 21 and a reflection plate 21a to measure the blood flow rate. Additionally, the transmitter 27 is accommodated in a casing 29, together with a battery 28 that is the power source of the blood flow rate probe 20. The casing 29 may be made of materials, such as polypropylene, silicone, having biocompatibility and allowing radio wave propagation therethrough. Each element of the blood flow rate probe 20 is fixed in the test body, by appropriate methods.

### Configuration of the Second Specific Example in the Third Embodiment

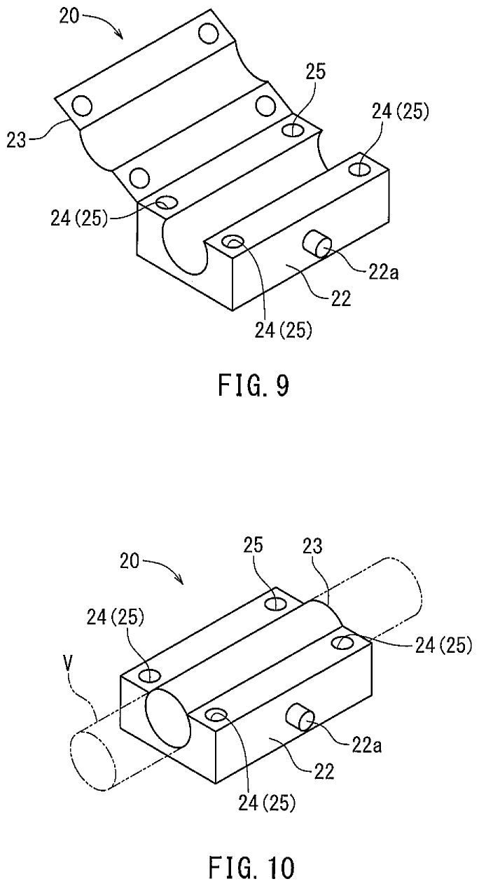

FIGS. 9 and 10 illustrate the second specific example of the third embodiment. Compared to the first specific example of the third embodiment, the second specific example of the third embodiment is characterized in only that the blood flow rate probe body 21 is replaced with another blood flow rate probe body 22. The other respects in both examples are substantially the same. Thus, the repetitive explanations or illustrations for the same elements are omitted. It is noted that the blood flow rate probe 20 shown in FIGS. 9 and 10 may also be used in the fourth embodiment described later.

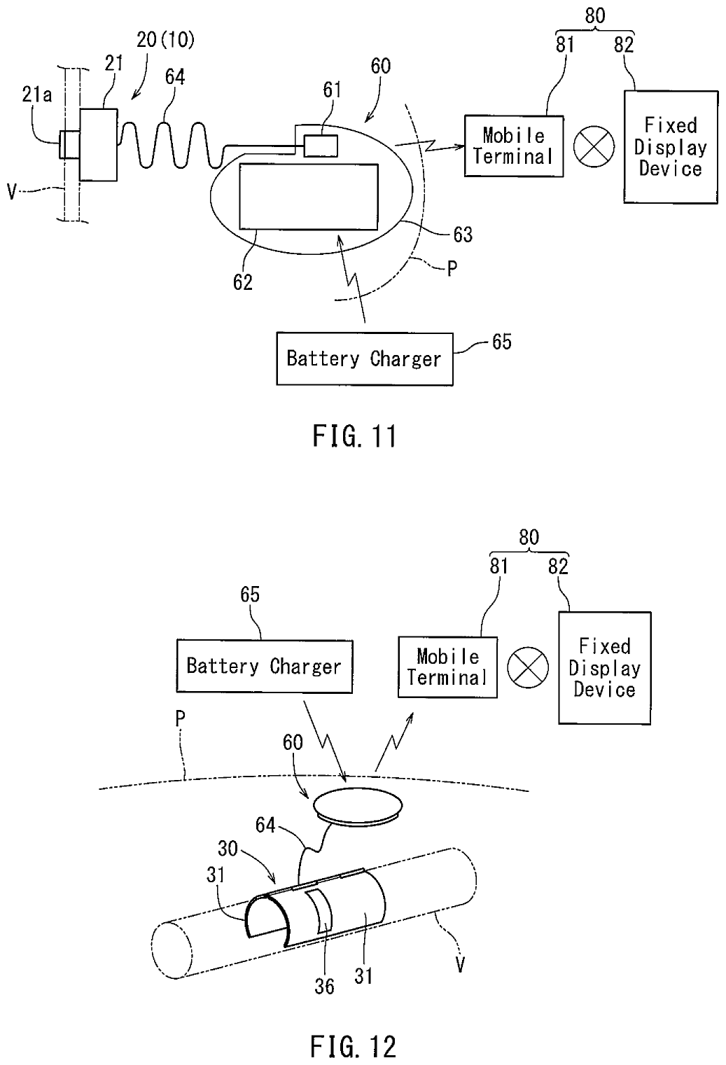

The blood flow rate probe body 22 adopts the TTFM to measure the blood flow rate, similar to that of the blood flow rate probe body 21. The blood flow rate probe body 22 may be covered by a lid 23. This allows the blood vessel V of the patient P to be placed within the blood flow rate probe body 22. Thus, the blood flow rate probe body 22 is configured to measure the blood flow rate. Measurement signals may be sent to the cord 26 (or the cord 64 in FIG. 11) via the connecter 22a (see FIGS. 8 and 11). The lid 23 may be fixed with screws 25 in a state so it covers the blood flow rate probe body 22. Some of the screws 25 can be used as electrodes for electrocardiogram. That is, the blood flow rate probe body 22 can also detect electrocardiograph signals at the same time.

### Function and Effect of the Third Embodiment

The blood flow rate and the electrocardiogram can be measured continuously for a certain period of time after an operation by using the blood flow rate probe 20 embedded in the body of the patient P.

### System Configuration in the Fourth Embodiment

FIG. 11 illustrates a system configuration in alignment with the fourth embodiment of the present disclosure. The fourth embodiment includes the blood flow rate probe 20 and a display device 80. The blood flow rate probe 20 can be used to measure the blood flow rate of the blood vessel V of the patient (test object) P and convert the measurement results into electric signals. The display device 80 can display the measured blood flow rates, so as to be viewable by healthcare professionals, including doctors. The blood flow rate probe 20 is configured to be embedded in the patient P for a certain period during or after an operation. The display device 80 may be placed at a position located outside the patient P where the healthcare professionals including the doctors, can easily see the display device 80.

The blood flow rate data obtained from the blood flow rate probe 20 is transmitted to a first transmitter 61 via a freely bendable cord 64. Then, the blood flow rate data is wirelessly transmitted from the first transmitter 61 to the display device 80. The freely bendable cord corresponds to an electric connecting member in the present disclosure. The first transmitter corresponds to a transmitter in the present disclosure. The distance between the blood flow rate probe 20 and the first transmitter 61 may change. However, the cord 64 connecting the blood flow rate probe 20 and the first transmitter 61 is freely bendable, and thus the cord 64 can flexibly adapt to the change of distance.

The first transmitter 61 is configured to be embedded in the patient P, in a state where the first transmitter 61 is accommodated in a casing 63. The casing 63 accommodates a battery 62, in addition to the first transmitter 61. The battery corresponds to a power source device in the present disclosure. The battery 62 may supply power to the first transmitter 61, and may also supply power to the blood flow rate probe 20, via the cord 64. The battery 62 may be recharged by a non-contact battery recharger 65 disposed outside the body. For example, an electromagnetic induction system or a radio wave receiving system may be used for charging the battery 62. However, a primary battery can be used as the battery 62, instead of the secondary battery, which is a rechargeable one. The first transmitter 61 may wirelessly communicate while still in the operating room. Thus, short distance communications such as Bluetooth®, may be used as the wireless communication method. The casing 63 may be made of, for example, a polypropylene resin or a silicone resin. Furthermore, the blood flow rate probe 20, the cord 64, the casing 63, and the other connecting members may have watertight and airtight structures allowing them to be buried in the body.

The mobile terminal 81, such as a mobile phone or a tablet terminal, may be used as the display device 80. The mobile terminal 81 has a built-in transceiver (not shown), which enables short distance communication as a standard function. Thus, the mobile terminal 81 may receive the blood flow rate data sent by the first transmitter 61 and display the blood flow rate on its own display. The mobile terminal 81 may continuously display the blood flow rate, although the mobile terminal 81 may display the blood flow rate at appropriate timing, such as intermittently.

In addition to the mobile terminal 81, a fixed display device 82 may be used as the display device 80. The fixed display device 82 can share the blood flow rate information with the mobile terminal 81 via wireless LAN. The fixed display device 82 may display, for example, an electrocardiogram, a body temperature, a blood pressure and a heart rate, which are measured separately, in addition to the blood flow rate. The mobile terminal 81 and the fixed display device 82 are not necessarily used at the same time. That is, only one of either of the mobile terminal 81 or the fixed display device 82 may be used. A plurality of the mobile terminals 81 and the fixed display devices 82 may be installed at different places and intercommunicate via wireless LAN.

The blood flow rate probe 20 (10) can be used to measure the blood flow rate using the TTFM, which is publicly known. When the blood flow rate is measured, the blood vessel V of the patient P is placed between the blood flow rate probe body 21 and the reflection plate 21a. However, the method for measuring the blood flow rate is not limited to the TTFM. The blood flow rate probe 20 can be used for other blood flow rate measuring devices adopting different methods.

### Function and Effect of the Fourth Embodiment

According to the fourth embodiment, the first transmitter 61 and the battery 62 are connected to the blood flow rate probe 20 (10) via the freely bendable cord 64. Further, the first transmitter 61 and the battery 62 may be separated from the blood flow rate probe 20 (10), so as to be located on the surface side of the patient P. Thus, if the blood vessel V is located in a narrow space in the body, only the blood flow rate probe 20 (10) can be inserted into the narrow space. This allows the first transmitter 61 and the battery 62 to be placed appropriately in a location separated from the blood flow rate probe 20 (10). As mentioned above, the first transmitter 61 and the battery 62 can be located on the surface side of the patient P, separated from the blood flow rate probe 20 (10). With this arrangement, a volume of a dielectric body (a portion of the patient P) within the communication path between the first transmitter 61 and the display device 80 can be reduced. This can improve the communication environment. Additionally, the first transmitter 61 and the battery 62 can be easily accessed from the surface side. Thus, maintenance of the first transmitter 61 and the battery 62 can be improved.

Furthermore, the first transmitter 61 and the battery 62 may be accommodated in a single casing 63. This allows the first transmitter 61 and the battery 62 to be handled as a single piece. Additionally, the first transmitter 61 and the battery 62 are covered by the casing 63. Thus, the first transmitter 61 and the battery 62 are protected from being exposed in the body.

### System Configuration in the Fifth Embodiment

FIG. 12 illustrates a system configuration in alignment with the fifth embodiment of the present disclosure. Compared to the fourth embodiment, the fifth embodiment is characterized in that the blood flow rate probe 20 (10) is replaced by another blood flow rate probe 30. The other respects, in the fourth and the fifth embodiments are substantially the same. Thus, the repetitive explanations or illustrations for the same elements are omitted.

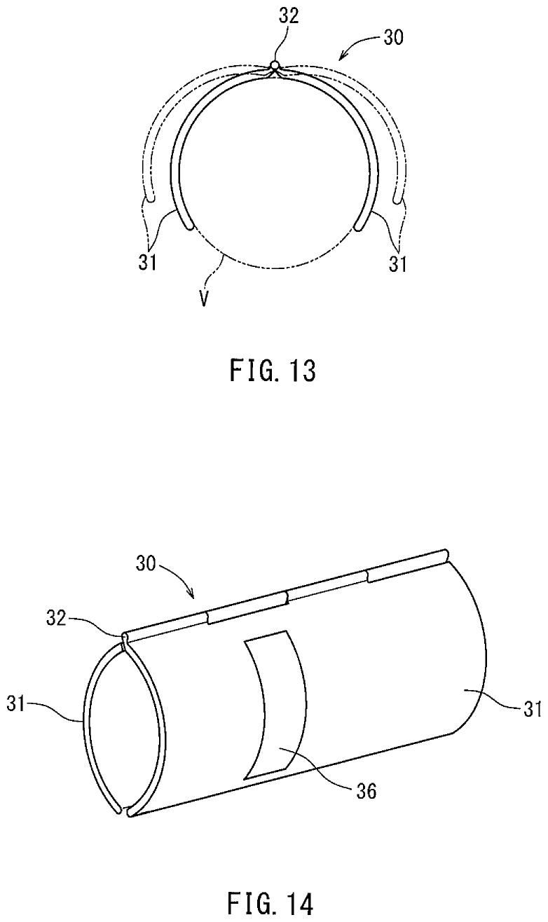

FIGS. 13 and 14 are enlarged views of the blood flow rate probe 30. The blood flow rate probe 30 has a pair of torso members 31 configured to be openable by a hinge member 32. The pair of the torso members 31 are made of, for example, a polypropylene resin. The pair of the torso members 31 have a curved shape so that the blood vessel V of the patient P can be placed between the torso members 31. When the blood vessel V is placed between the pair of the torso members 31, the torso members 31 are spaced apart from each other, as shown by the imaginary lines in FIG. 13. After the blood vessel is inserted between the pair of torso members 31, the torso members 31 are closed toward each other so that the torso members 31 can fit the blood vessel V therebetween, as shown by solid lines in FIG. 13. When the blood vessel V is placed between the pair of the torso members 31, as shown by the solid lines in FIG. 13, the torso members 31 form a partially opened circular pipe shape that allows the blood vessel V to pass therethrough. With this arrangement, the pair of torso members 31 can wrap around an outer peripheral surface of the blood vessel V.

In this way, the pair of the torso members 31 is configured to be openable about the hinge member 32. Thus, it is easy to place the blood vessel V between the pair of torso members 31. The operator can open the pair of torso members 31 with their fingers, via the hinge member 32. Even if the operator does not touch the torso members 31, a frictional force applied between the pair of the torso members 31 keeps the relative angle between the torso members 31.

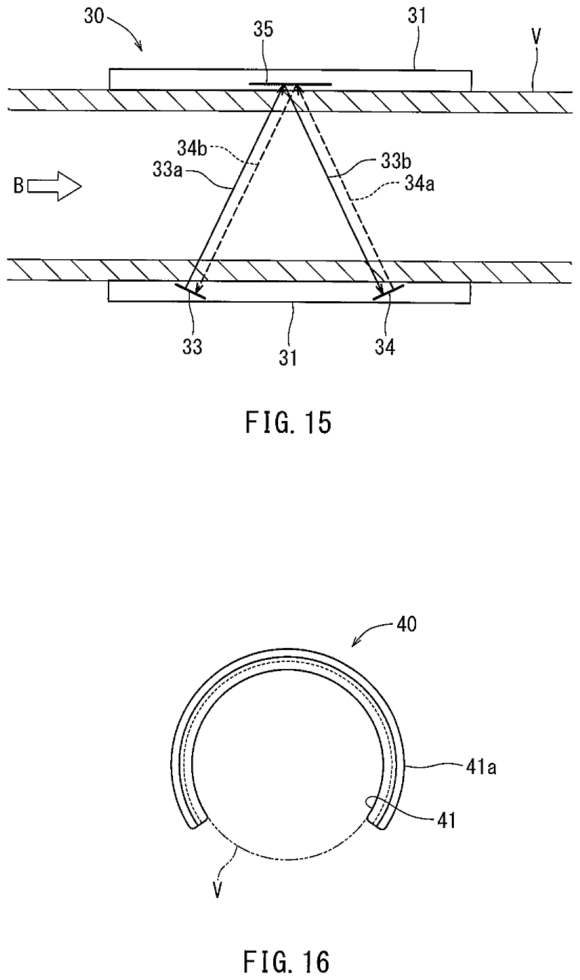

FIG. 15 illustrates a principle of measuring the blood flow rate with the blood flow rate probe 30. The blood flow rate probe 30 may adopt the publicly known TTFM to measure the blood flow rate. One torso member 31 has a reflection plate 35 on its inner wall surface. The other torso member 31 has a first transducer 33 and a second transducer 34 on its inner wall surface. The first and the second transducers 33, 34 are opposite to the reflection plate 35. The reflection plate 35 reflects ultrasonic waves 33a, 34a respectively emitted from the first and the second transducers 33, 34 in a state that the blood vessel V is placed between the reflection plate 35 and the first and the second transducers 33, 34. Then, the first and the second transducers 33, 34 receive the reflected ultrasonic waves 34b, 33b, respectively. The torso member 31 has a processing circuit 36 positioned at its outer wall (e.g., see FIG. 14). The processing circuit 36 can determine a flow rate of a blood flow B in the blood vessel V based on time lags between transmission and receipt of the ultrasonic waves in the first and the second transducers 33, 34. The measurement results of the blood flow rates by the blood flow rate probe 30 are converted to electric signals. Then, the electric signals are sent to a transmitter unit 60 via the cord 64, as shown in FIG. 12.

To measure the blood flow rate with the blood flow rate probe 30, the reflection plate 35 needs to reflect the ultrasonic waves 33a, 34a respectively emitted from the first and the second transducers 33, 34. Additionally, the first and the second transducers 33, 34 need to receive the reflected ultrasonic waves 34b, 33b respectively. Thus, the relative angle between the pair of the torso members 31 needs to be maintained, so that the blood flow rate can be measured. Thus, the blood flow rate probe 30 may have different sizes depending on the diameters of the blood vessel V which is the target to be measured. In this way, the relative angles between the pair of the torso members 31 can be maintained to measure the blood flow rate.

[Another Example of the Blood Flow Rate Probe 30]

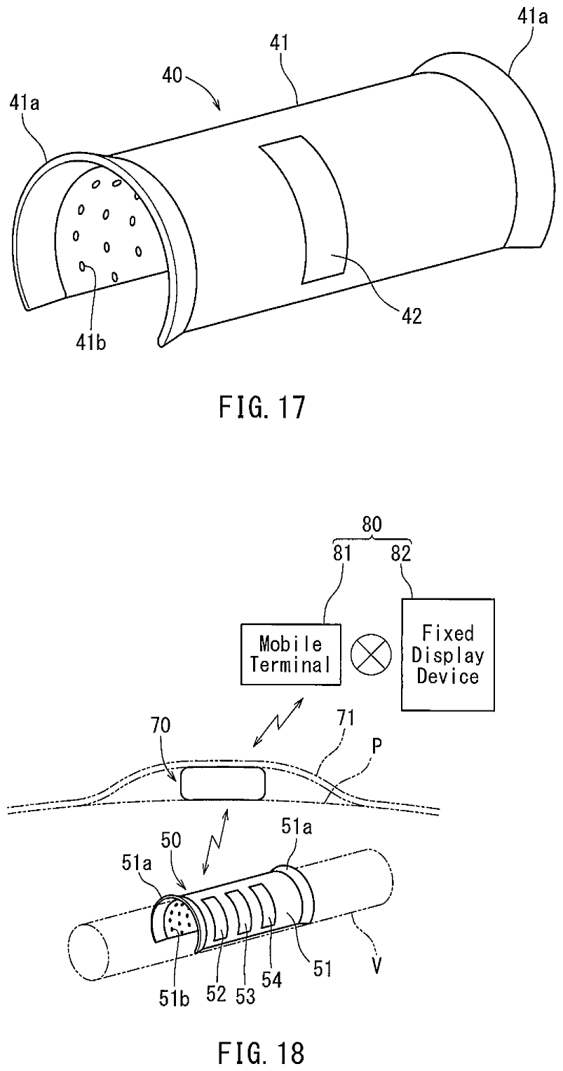

FIGS. 16 and 17 illustrate another example of the blood flow rate probe 30 in the fifth embodiment. A blood flow rate probe 40 is different from the blood flow rate probe 30. The main difference is that the blood flow rate probe 40 has a torso member 41 having an integrated structure with no hinge member 32. The torso member 41 has diameter-expanded portions 41a at both edges (e.g., see FIG. 17). Each of the diameter-expanded portions 41a has a shape that expands gradually toward an edge side of the torso member 41. The torso member 41 may have a number of small projections 41b at its inner wall, which may increase frictional resistance between the inner wall and the blood vessel V. The blood flow rate probe 40 can be used to measure the blood flow rate by the TTFM, similar to the blood flow rate probe 30. The blood flow rate probe 40 may have a processing circuit 42 at its outer wall, similar to the processing circuit 36 of the blood flow rate probe 30.

The blood flow rate probe 40 is not configured to be openable in the same way as the blood flow rate probe 30. Instead, the blood vessel V can be inserted into the torso member 41 by deforming the blood vessel V into an elliptical shape. However, the torso member 41 may also be elastically deformed to slightly widen its opening when the blood vessel V is inserted into the torso member 41. The blood flow rate probe 40 may be prepared in various sizes, depending on the diameters of the blood vessels V to be measured.

The blood flow rate probe 40 has a diameter-expanded portions 41a at each of its edges. The diameter-expanded portions 41a can prevent the both edges of the torso member 41 from digging into the outer peripheral surface of the blood vessel V, for instance when the blood vessel V is inserted into the torso member 41. Furthermore, the torso member 41 has a number of projections 41b at its inner wall. This can prevent the torso member 41 holding the blood vessel V, from slipping along the surface of the blood vessel V, for instance due to the gravity when the blood vessel V is in a vertical or substantially vertical state.

### Function and Effect of the Fifth Embodiment

According to the fifth embodiment, the curved shape torso members 31, 41 can wrap around the outer peripheral surface of the blood vessel V for which the blood flow rate is being measured. The torso members 31, 41 can be also attached easily to the blood vessel V located in a narrow space. In this way, the torso members 31, 41 can be used for various blood vessels V.

### System Configuration in the Sixth Embodiment

FIG. 18 illustrates a system configuration according to the sixth embodiment of the present disclosure. In the fifth embodiment, the torso members 31, 41 send the measured data to a transmitter unit 60 via a cord 64 (e.g., see FIG. 12). On the other hand, the sixth embodiment is characterized in that a torso member 51 send measured data to a transmitter unit 70 via wireless communication. The other configurations in the sixth embodiment are substantially the same as the fifth embodiment. Accordingly, the same symbols are attached to the same elements. Thus, repetitive explanations for the same elements are omitted.

The torso member 51 of the blood flow rate probe 50 in the sixth embodiment has a configuration similar to the torso member 41 in the fifth embodiment. The difference between them is only a configuration to connect the transmitter unit 70 with the torso member 51 via wireless communication. The other configurations may be substantially the same. The torso member 51 may also have diameter-expanded portions 51a and projections 51b that are substantially the same as the diameter-expanded portions 41a and the projections 41b of the torso member 41.

The torso member 51 has a power supply equipment 52, a processing circuit 53, and a second transmitter at its outer peripheral surface. The second transmitter corresponds to a transmitter in the present disclosure. The power supply equipment 52 is wirelessly fed power from a power source device (not shown) accommodated in the transmitter unit 70. The fed power supplies working power to the processing circuit 53 and the second transmitter 54. The wireless power feeding may adopt, for example, a feeding method using radio waves. The wireless power feeding corresponds to an electric connection means of the present disclosure. The processing circuit 53 can be used to measure the blood flow rate based on the time lags between transmission and receipt of the ultrasonic waves, for instance as determined by the TTFM used by the processing circuit 42 in the fifth embodiment. The second transmitter 54 may transmit the blood flow rate data determined by the processing circuit 53 to a first transmitter (not shown) accommodated in the transmitter unit 70. The second transmitter 54 may communicate with the first transmitter via in vivo communication. The transmitter unit 70 may be attachable to an outside the surface of the patient P, for instance by a belt 71. The second transmitter 54 may be able to communicate directly with the mobile terminal 81, without using the first transmitter of the transmitter unit 70, depending on certain conditions, such as a position of the blood vessel V in the patient P.

### Function and Effect of the Sixth Embodiment

According to the sixth embodiment, the blood flow rate probe 50 embedded in the patient P's body can be used to measure blood flow rate. The measured data is sent from the second transmitter 54 to the display device 80 located outside of the patient P's body via the first transmitter, which is in the transmitter unit 70, by wireless communication. Then, the data is displayed on the display device 80. The power source device, which is provided in the transmitter unit 70, supplies electric power to the blood flow rate probe 50, via the power supply equipment 52. The transmitter unit 70 may be fixed to the outside of the surface of the patient P. Thus, only the blood flow rate probe 50 is embedded in the patient P. This can decrease the physical burden during the measurement of the blood flow rate on the patient P. Thus, the blood flow rate measurement system of the sixth embodiment allows the measurement to continue for a relatively long term, such as several months, after the operation. The torso member 51 of the blood flow rate probe 50 has the power supply equipment 52. The power supply equipment 52 may be mainly used to stabilize the power supply when receiving the electric energy from the power source device of the transmitter unit 70. Thus, the power supply equipment 52 may not need to keep supplying the accumulated electricity for a long time. Accordingly, the size of the blood flow rate probe 50 may only be slightly bigger than a blood flow rate probe having a normal power source device.

### The Other Embodiment

While the present disclosure has been described herein with reference to specific embodiments, various kinds of changes, additions, or deletion may be possible, for example, but are not limited to their appearances or structures. For example, the blood flow rate measurement system of the present disclosure may be applied to not only human blood vessels, but also animal blood vessels. Additionally, the blood flow rate measurement system of the present disclosure may be applied to not only blood vessels of the heart, but also blood vessels of the other organs of the test body. Furthermore, the blood flow rate measurement system of the present disclosure may be applied to, for example, artificial hearts or artificial blood vessels.

1. A blood flow rate measurement system, comprising:
a blood flow rate probe configured to measure a blood flow rate of a blood vessel of a test body and to convert the blood flow rate to an electric signal; and
a display device configured to display the blood flow rate measured by the blood flow rate probe;
wherein data of the blood flow rate is transmitted from the blood flow rate probe to the display device via wireless communication,
wherein the blood flow rate probe comprises a monitor configured to display a part of the blood flow rate data displayed on the display device, and
wherein the part of the blood flow rate data is transmitted from the display device to the blood flow rate probe via wireless communication.

2. A blood flow rate measurement system, comprising:
a blood flow rate probe configured to measure a blood flow rate of a blood vessel of a test body and to convert the blood flow rate to an electric signal; and
a display device configured to display the blood flow rate measured by the blood flow rate probe,
wherein data of the blood flow rate is transmitted from the blood flow rate probe to the display device via wireless communication.

3. The blood flow rate measurement system according to claim 2, wherein the blood flow rate probe comprises a monitor configured to display a part of the blood flow data.

4. The blood flow rate measurement system according to claim 2, wherein the blood flow rate probe is embedded and fixed in the test body for a certain period of time.

5. An internally implantable blood flow rate measurement system, comprising:
a blood flow rate probe embedded in a test body, and attached to a blood vessel of the test body, the blood flow rate probe being configured to convert a blood flow rate to an electric signal;
a display device located outside of the test body, the display device being configured to display the blood flow rate measured by the blood flow rate probe;
a transmitter configured to transmit data of the blood flow rate to the display device via wireless communication after receiving the electric signal from the blood flow rate probe; and
a power source device configured to supply working power to the blood flow rate probe and to the transmitter,
wherein the power source device is connected to the blood flow rate probe via an electric connection device allowing to change connecting distance so that the power source device is configured to be located on a surface side of the test body apart from the blood flow rate probe.

6. The internally implantable blood flow rate measurement system according to claim 5,
wherein the power source device is configured to be embedded in the test body in a state that the power source device is accommodated in a casing, and
wherein the electric connection device comprises of a freely bendable cord.

7. The internally implantable blood flow rate measurement system according to claim 5,
wherein the power source device is located outside of the test body,
wherein the blood flow rate probe has power supply equipment wirelessly being fed from the power source device, and
wherein the power supply equipment is configured to supply working power to the blood flow rate probe and the transmitter.

8. The internally implantable blood flow rate measurement system according to claim 5, wherein the blood flow rate probe comprises:
a torso member configured to be attachable to the blood vessel by wrapping around an outer peripheral surface of the blood vessel to be measured,
wherein the torso member has a circular pipe shape partially opened, such that the blood vessel is allowed to be inserted into the circular pipe shape, and
wherein the torso member is configured to measure the blood flow rate of the blood vessel.

9. The internally implantable blood flow rate measurement system according to claim 8, wherein the torso member is separated into a first portion and a second portion about a hinge member.

10. The internally implantable blood flow rate measurement system according to claim 8, wherein the torso member further comprises a plurality of projections projecting from an internal surface configured to face the blood vessel.

11. The internally implantable blood flow rate measurement system according to claim 8, wherein the torso member further comprises a reflection plate formed on a first side of the torso member and a first and second transducers formed on a second side of the torso member opposite the first side.

12. The internally implantable blood flow rate measurement system according to claim 8,
wherein the blood flow rate probe further comprises a diameter expanded portion formed at one side of the torso member, and
a diameter of the diameter expanded portion is larger than a diameter of the torso member.

13. The internally implantable blood flow rate measurement system according to claim 5, wherein the blood flow rate probe further comprises:
a body having an opening into which the blood vessel may be placed; and
a lid configured to cover the opening of the body and maintain a relative distance to the body.

14. The internally implantable blood flow rate measurement system according to claim 13,
wherein the lid is fixed to the body by a fixing member, and
wherein the fixing member is configured to be an electrode for generating an electrocardiogram.

15. The internally implantable blood flow rate measurement system according to claim 5, wherein a distance between the blood flow rate probe and the surface of the test body is greater than a distance between the transmitter and the surface of the test body.
